(19) United States
(12) Patent Application Publication
(10) Pub. No.: US 2026/0345700 A1
(43) Pub. Date: Nov. 5, 2026

(54) TOPICAL FORMULATION COMPRISING DELIQUESCENT SUBSTANCE

(71) Applicant: L'Oreal S.A., Paris (FR)

(72) Inventors: Charlotte Vedel, Copenhagen (DK); Anders Bech Bruntse, Copenhagen (DK); Soren Kjaerulff, Copenhagen (DK)

(73) Assignee: L'Oreal S.A., Paris (FR)

(21) Appl. No.: 19/487,292

(22) PCT Filed: Nov. 22, 2023
(86) PCT No.: PCT/EP2023/082735

§ 371 Date: Nov. 24, 2025

(30) Foreign Application Priority Data
May 30, 2023 (DK) PA 2023 30051
Jul. 3, 2023 (DK) PA 2023 30112
Oct. 11, 2023 (DK) PA 2023 30256

(51) Int. Cl. A61K 35/747; A61K 8/20; A61K 8/23; A61K 8/92; A61K 8/99; A61K 35/00; A61K 35/744; A61K 47/02; A61K 47/22; A61K 47/44; A61Q 19/00

(52) U.S. Cl. CPC A61K 35/747; A61K 8/20; A61K 8/23; A61K 8/922; A61K 8/99; A61K 35/744; A61K 47/02; A61K 47/22; A61K 47/44; A61Q 19/00; A61K 2035/115; A61K 2800/591

(57) ABSTRACT

The present invention relates to a topical anhydrous composition comprising an anhydrous substance, at least one viable microorganism and a deliquescent substance.

### TECHNICAL FIELD OF THE INVENTION

The present invention is related to a new composition, a new method of making stable formulations and a new use of deliquescent substances. Products based thereon are stable over prolonged periods of time and maintain the viability of microorganisms in the product.

The invention has particular advantage when stabilizing live microorganisms in an anhydrous substance or essential anhydrous substance for topical application of microorganisms.

The present invention relates to water free or essential water free systems comprising viable microorganisms. In particular, the present invention relates to compositions for topical use on skin or mucous membranes comprising at least one viable microorganism.

In particular, the present invention relates to stability of viable microorganisms in cosmetic or pharmaceutical products for topical application.

### BACKGROUND OF THE INVENTION

There is considerable interest in the use of probiotic bacteria. Probiotics are live microorganisms that confer health benefits to the host when administered at adequate levels (FAO WHO, 2006). However, to exert these benefits, the microorganisms must remain viable during the processing and storage of the product containing live probiotics. Considerable amount of research has been done to stabilize probiotics for oral consumption and ensure resistance to gastrointestinal fluids.

In order to meet the demand of skin care products comprising live microorganisms, it is necessary to develop stable compositions for topical use which can maintain viability of the microorganisms as well as secure activation of the microorganism when applied on skin or mucous membranes.

Topical formulations and products for pharmaceutical or cosmetic purposes are developed to have a long shelf life at room temperature and to be stable towards contamination and spoilage caused by microorganisms. The stability of viable probiotics in these topical formulations are very limited, however, the use of probiotics in topical formulations could have a huge potential if viability can be maintained in the formulation. Topical formulations like creams, lotions, gels, and mists inherently contain a high degree of water, i.e. in order to be suitably formulated into a gel, cream, foam, lotion, ointment etc. Evidently, the presence of such high degrees of water in these formulations, poses a problem for the storage of probiotics in their metabolically inactive condition. A second problem occurring in such topical formulations, is that these generally contain agents, which are not compatible with the survival of microorganisms; such as preservatives, surfactants, emulsifiers and other ingredients in order to protect such formulations against the growth of unwanted microorganisms as well as for forming stable emulsions.

The major problem observed when formulating live probiotic strains in gels, emulsions, lotions and the like for topical application is lack of viability at long term storage.

Hence, it was an object of the present invention to provide a composition allowing for long-term storage of viable microorganisms, which does not substantially harm such microorganisms upon use thereof and which does release the viable microorganisms when applied on the skin or on mucous membranes.

### SUMMARY OF THE INVENTION

Thus, an object of the present invention relates to anhydrous topical compositions with increased stability comprising viable microorganisms and a deliquescent substance, preferably an inorganic salt, use of such composition and a method of preparing such composition.

In particular, it is an object of the present invention to provide an anhydrous topical composition where stability of live microorganisms, in particular added live microorganisms, may be improved.

An aspect of the present invention relates to a topical anhydrous composition comprising an anhydrous substance, at least one viable microorganism and a deliquescent substance.

A further aspect of the present invention relates to a topical anhydrous composition comprising at least one viable microorganism and a deliquescent substance wherein the weight ratio of the deliquescent substance to the viable microorganism is in the range from 10:1 to 1:10.

Yet an aspect of the present invention relates to the use of a composition according to the present invention on skin or mucous membranes or genitals of a mammal.

A further aspect of the present invention relates to a composition comprising the topical anhydrous composition according to the present invention in a cosmetic, in a prophylaxis medicament or in a medicament, for the treatment of a disease, dysfunction or disorder.

Still a further aspect of the present invention relates to the use of a deliquescent substance to stabilize viable microorganisms in an anhydrous topical composition.

Still a further aspect of the present invention relates to a method for providing a topical anhydrous composition comprising an anhydrous substance, at least one viable microorganism and a deliquescent substance wherein the deliquescent substance is subjected to a step of dehydration or partly dehydration before being mixed into the anhydrous substance providing the anhydrous composition.

Yet an aspect of the present invention relates to a process for producing an anhydrous composition, the method comprises the steps of:
Providing an anhydrous substance;
Add a deliquescent substance, like a deliquescent salt to the anhydrous substance;
Homogenize the mixture of anhydrous substance and deliquescent substance;
Optionally, cool the melted anhydrous substance comprising the deliquescent substance, like a deliquescent salt, to a temperature below approximately 30 °C:
Add a at least one probiotic microorganism; and
homogenize the mixture providing the anhydrous composition.

A further aspect of the present invention relates to an anhydrous composition may be used for topical application to human skin.

A further aspect of the present invention relates to an anhydrous composition may be used for topical application to human mucous membranes.

A further aspect of the present invention relates to an anhydrous composition may be used for application to mucous membranes of the genitals.

A further aspect of the present invention relates to an anhydrous composition may be used for topical cosmetic applications.

A further aspect of the present invention relates to an anhydrous composition may be used for topical pharmaceutical applications.

A further aspect of the present invention relates to an anhydrous composition may be used for a cosmetic, a prophylaxis medicament or a medicament for the treatment of a disease, dysfunction, or disorder.

A further aspect of the present invention relates to an anhydrous composition may be used for a cosmetic for anti-age treatment.

The present invention will now be described in more detail in the following.

### DETAILED DESCRIPTION OF THE INVENTION

It was surprisingly found that formulating the microorganisms in an anhydrous substance with a deliquescent substance, like a deliquescent water-soluble salt, may significantly improve stability and shelf life of the live microorganism in the anhydrous composition.

Inorganic salts are well known to be antimicrobial and are traditionally used for preservation and inhibition of microorganisms, especially with a long history as preservatives being used in the food industry. It was therefore unexpected to find that these water-soluble salts have a stabilizing effect on viable microorganisms in anhydrous compositions, thus being able to significantly extend the viability of microorganisms and prolong the storage stability of such products.

Water-soluble inorganic salts are used in the cosmetic and pharmaceutical industry in multiple water-based topical products as a binder, mild abrasive, thickener and preservative, e.g. as viscosity increasing agents to enhance texture of cosmetic products, often combined with guar-based, cellulose, or gum-based thickeners. Water-soluble salts are typically not used in anhydrous (water free) products e.g. oils and fats used as topical cosmetic. The antimicrobial activity of inorganic salt was not observed in anhydrous compositions and even more surprisingly it was observed that the inorganic salt was able to stabilize the viable (live) microorganisms and improve long term viability of the microorganisms.

The antimicrobial activity of MgCl₂ (magnesium chloride) may show to be much stronger than the that of NaCl (sodium chloride) or KCl (potassium chloride) therefore even more surprisingly it was found that MgCl₂ stabilised the survival and viability of microorganisms in anhydrous (water free) compositions, in a stronger manner compared to NaCl or KCl.

### DEFINITIONS

Hygroscopic substances are substances that attract water molecules from the air without necessarily dissolving in the absorbed water and reach a state of equilibrium with the humidity around them. This is due to the presence of hydrophilic groups on the surface of the solid. Deliquescent substances are substances that absorb and dissolve in water to form a liquid. This is due to the absorption of water molecules by the solid.

Deliquescence, like hygroscopy, is also characterized by a strong affinity for water and tendency to absorb moisture from the atmosphere or the surrounding environment if exposed to it. Unlike hygroscopy, however, deliquescence involves absorbing sufficient water to form an aqueous solution.

“Deliquescent substance”, within the meaning of the invention, is a salt characterized by the dehydrated substance ability to adsorb water below 35 °C, more preferably below 30 °C, more preferably below 25 °C, more preferably between 15 °C and 25 °C.

Thus, preferably, the deliquescent substance is a salt that adsorbs water at 25 °C.

Within the meaning of the invention, typically, the absorption of water increases the weight of deliquescent substance by at least 5% (w/w), calculated as by weight of the total weight of the adsorbed deliquescent substance (deliquescent substance including absorbed water) by the weight of the deliquescent substance (w/w), more preferably by at least 15% (w/w), more preferably by at least 30% (w/w), more preferably by at least 50% (w/w).

Absorption of water can be determined by standard procedures for water absorption. An example hereof is:
1. The deliquescent substance is dried in an oven for a specified time and temperature to remove water;
2. Then it is placed in a desiccator or airtight container to cool;
3. Immediately upon cooling the deliquescent substance is weighted;
4. The deliquescent substance is then placed in open air or at a chosen relative humidity, and water absorption is measured and followed over time until the weight increase stabilizes at a stationary level; and
5. The weight is measured, and the weight increase is calculated as percentage increase of the weight measured in point 3.

Within the meaning of the invention, the deliquescent substance absorbs water below 99% relative humidity, more preferably below 90% relative humidity, more preferably below 80% relative humidity, more preferably below 60% relative humidity, more preferably below 40% relative humidity at 25 °C.

Humidity can be measured using a hygrometer, i.e. a tool that utilizes various materials and measurements to gauge a room or spaces level of water vapor. Relative humidity is the amount of water vapor in air compared to the amount of water vapor that the air can hold. The amount of water vapor that air can hold depends on the temperature; the warmer the air, the more water vapor it can hold. Air is said to be saturated when it cannot hold any more water. Multiple standard instruments are available to determine relative humidity.

Preferably, the deliquescent substance may be a salt selected from a water soluble inorganic salt or a water soluble organic salt. preferably, the salt may be a water soluble inorganic salt.

In an embodiment of the present invention the deliquescent substance may be a deliquescent salt. Preferably the deliquescent salt may be selected from the group consisting of calcium chloride (CaCl₂), magnesium chloride (MgCl₂), zinc chloride (ZnCl₂), ferric chloride (FeCl₃), lithium chloride (LiCl), carnallite (KCl·MgCl₂), potassium carbonate (K₂CO₃), sodium acetate anhydrous (CH₃COONa), ammonium nitrate (NH₄NO₃), potassium hydroxide (KOH), dipotassium phosphate (K₂HPO₄), magnesium nitrate (Mg(NO₃)₂), sodium formate (NaCHO₂), sodium acetate (NaC₂H₃O₂), trisodium phosphate (Na₃PO₄), magnesium sulfate (MgSO₄), sodium sulfate (Na₂SO₄), aluminum potassium sulfate (CH₃NH₂Al(SO₄)₂), Manganese(II) chloride (MnCl₂), aluminum sulphate (Al₂(SO₄)₃), aluminum chloride (AlCl₃), cobalt(II) chloride (CoCl₂), sodium alum, NaAl(SO₄)₂, sodium hydroxide (NaOH), and a combination hereof.

Preferably the deliquescent salt may be a water soluble salt selected from magnesium chloride (MgCl₂), calcium chloride (CaCl₂), carnallite (KCl·MgCl₂), potassium carbonate (K₂CO₃), sodium acetate anhydrous (CH₃COONa), and dipotassium phosphate (K₂HPO₄) and any combinations hereof.

In another embodiment of the invention, the deliquescent substances may be a partly deliquescent substance and have deliquescent behaviour when applied to skin. Examples of such partly deliquescent substance may be sodium chlorite (NaClO₂) or ozalic acid (H₂C₂O₄).

The present invention relates to deliquescent substances for anhydrous topical compositions for mammals, therefore some of the listed deliquescent substances e.g. zinc chloride, lithium chloride and ferric chloride may not be suited or desired due to toxicity. In an embodiment of the present invention the deliquescent substances may be selected from the group comprising of calcium chloride (CaCl₂), magnesium chloride (MgCl₂), carnallite (KCl·MgCl₂), potassium carbonate (K₂CO₃), sodium acetate anhydrous (CH₃COONa), potassium hydroxide (KOH), dipotassium phosphate (K₂HPO₄) and sodium hydroxide (NaOH) or any combinations thereof.

The human skin has an acid pH, and therefore most topical compositions intent to have an acidic or slightly acidic pH, a deliquescent substance supporting the natural pH of healthy skin will be advantageous. In a preferred embodiment of the present invention the deliquescent substance may be selected from calcium chloride (CaCl₂), magnesium chloride (MgCl₂), carnallite (KCl·MgCl₂), potassium carbonate (K₂CO₃), sodium acetate anhydrous (CH₃COONa), dipotassium phosphate (K₂HPO₄), or a combination hereof.

Preferably, the deliquescent substances may be selected from calcium chloride (CaCl₂), magnesium chloride (MgCl₂), carnallite (KCl·MgCl₂), potassium carbonate (K₂CO₃), sodium acetate anhydrous (CH₃COONa), dipotassium phosphate (K₂HPO₄), or any combinations hereof.

Preferably the deliquescent substance is selected from from calcium chloride (CaCl₂), magnesium chloride (MgCl₂), carnallite (KCl·MgCl₂), potassium carbonate (K₂CO₃), dipotassium phosphate (K₂HPO₄) and their combinations.

Even more preferably, the deliquescent substance may comprise a magnesium salt, in particular magnesium chloride (MgCl₂).

The deliquescent substance may be a salt hydrate which can be dehydrated to form an unstable hydration level in the salt at temperature from 5-40° c.

In an embodiment of the present invention the deliquescent substance may be a salt hydrate which at standard atmospheric pressure and relative humidity between 45% and 75% will be fully hydrated or partly hydrated. The deliquescent salt according to the present invention may dehydrate at a temperature above 20 °C. More preferably the deliquescent salt according to the present invention may dehydrate at a temperature above 25 °C. More preferably the deliquescent salt according to the present invention may dehydrate at a temperature above 30 °C. More preferably the deliquescent salt according to the present invention may dehydrate at a temperature above 40 °C. More preferably the deliquescent salt according to the present invention may dehydrate at a temperature above 50 °C.

In yet an embodiment of the present invention the deliquescent substance according to the present invention may be a dry deliquescent substance. Preferably, the dry deliquescent substance comprises a moisture content below 8% (w/w), e.g. below 6% (w/w), such as below 4% (w/w), e.g. below 2% (w/w), such as below 1% (w/w), e.g. below 0.5% (w/w), such as below 0.1% (w/w), e.g. below 0.05% (w/w), such as below 0.01% (w/w),

The deliquescent substance and the viable microorganisms may be added to the anhydrous substance providing the anhydrous composition.

A preferred embodiment of the present invention relates to a topical anhydrous composition comprising an anhydrous substance, at least one viable microorganism, and a deliquescent substance.

In an embodiment of the present invention the deliquescent substance may be placed in the anhydrous substance. Preferable, the deliquescent substance may be distributed in the anhydrous substance. Even more preferable, the deliquescent substance may be homogenously, or substantially homogenously, distributed in the anhydrous substance.

In an embodiment of the present invention the deliquescent substance may be represent in the range of 0.05 to 5% (w/w) of the anhydrous composition, more preferable in the in the range of 0.075 to 4% (w/w), more preferable in the in the range of 0.1 to 2% (w/w), more preferable in the in the range of 0.15 to 1.75% (w/w), more preferable in the range of 0.2 to 1.5% (w/w).

The deliquescent substance, e.g. the deliquescent salt may be dried and distributed in the anhydrous substance as crystals, e.g. salt crystals. Preferably the salt crystals may have a size less than 500 μm, such as less than 400 μm, e.g. less than 300 μm, such as less than 200 μm, e.g. less than 100 μm, such as less than 75 μm, e.g. less than 50 μm, e.g. less than 30 μm, e.g. less than 10 μm.

The weight ratio of the deliquescent substance to the viable microorganism is in the range from 10:1 to 1:10, such as in the range of 8:1 to 1:8, e.g. in the range of 6:1 to 1:6, such as in the range of 4:1 to 1:4, e.g. in the range of 2:1 to 1:2, e.g. in the range of 2:3 to 1:3, such as about 1:1.

The present invention relates to and anhydrous composition comprising live microorganisms including any bacteria, archaea, phages, viruses, yeast or fungi or any combinations thereof.

In an embodiment of the present invention the live microorganism may be a probiotic live microorganism.

Examples of suitable probiotic microorganisms include bacteria such as the genera Bifidobacterium, Bacteroides, Clostridium, Fusobacterium, Melissococcus, Propionibacterium, Streptococcus, Enterococcus, Lactococcus, Staphylococcus, Peptostrepococcus, Bacillus, Pediococcus, Micrococcus, Leuconostoc, Weissella, Aerococcus, Oenococcus, Cutibacterium, Lactobacillus, Lactiplantbacillus, Holzapfelia, Amylolactobacillus, Bombilactobacillus, Companilactobacillus, Lapidilactobacillus, Agrilactobacillus, Schleiferilactobacillus, Loigolactobacillus, Lacticaseibacillus, Latilactobacillus, Dellaglioa, Liquorilactobacillus, Ligilactobacillus, Furfurilactobacillus, Paucilactobacillus, Limosilactobacillus, Fructilactobacillus, Acetilactobacillus, Apilactobacillus, Levilactobacillus, Secundilactobacillus, Lentilactobacillus, Aerococcus, Carnobacterium, Enterococcus, Oenococcus, Sporolactobacillus, Nitrosomonas, Tetragenococcus, Vagococcus, and Weissella as well as yeasts such as Saccharomyces, Debaromyces, Candida, Pichia and Torulopsis, moulds such as Aspergillus, Rhizopus, Mucor, and Penicillium and Torulopsis.

The most commonly used probiotics are strains of the lactic acid bacteria (LAB).

These LAB are considered non-pathogenic and are used as probiotic bacteria in general to improve gastrointestinal flora and in the treatment of gastrointestinal symptoms. The present invention may relate to stabilization of any viable microorganisms in an anhydrous composition for topical application. The bacteria are preferably selected among the genera Lactobacillus, Lactiplantbacillus, Holzapfelia, Amylolactobacillus, Bombilactobacillus, Companilactobacillus, Lapidilactobacillus, Agrilactobacillus, Schleiferilactobacillus, Loigolactobacillus, Lacticaseibacillus, Latilactobacillus, Dellaglioa, Liquorilactobacillus, Ligilactobacillus, Furfurilactobacillus, Paucilactobacillus, Limosilactobacillus, Fructilactobacillus, Acetilactobacillus, Apilactobacillus, Levilactobacillus, Secundilactobacillus, Lentilactobacillus, Leuconostoc, Bifidobacterium, Pediococcus, Lactococcus, Streptococcus, Aerococcus, Carnobacterium, Enterococcus, Oenococcus, Sporolactobacillus, Tetragenococcus, Staphylococcus, Cutibacterium, Probionibacterium, Vagococcus, and Weissella.

The preferred microorganisms may be bacteria, in particular probiotic bacteria. The probiotic bacteria may preferably be selected from the group comprising Lactococcus lactis, Lacticaseibacillus rhamnosus, Lactiplantibacillus plantarum, Lactobacillus helveticus, Lactobacillus jensenii, Lactobacillus acidophilus, Lactobacillus bulgaricus, Lactobacillus amylovorus, Lactobacillus amylolyticus, Lactobacillus alimentarius, Lactobacillus aviaries, Lactobacillus delbrueckii, Lactobacillus diolivorans, Lactobacillus farciminis, Lactobacillus gallinarum, Lacticaseibacillus casei, Lactobacillus crispatus, Lactobacillus gasseri, Lactobacillus johnsonii, Lactobacillus hilgardii, Lactobacillus kefiranofaciens, Lactobacillus kefiri, Lactobacillus mucosae, Lactobacillus panis, Lactiplantibacillus paraplantarum, Lactobacillus pontis, Latilactobacillus sakei, Lactobacillus saliverius, Lactobacillus sanfraciscensis, Lacticaseibacillus paracasei, Lactobacillus pentosus, Lactobacillus cellobiosus, Lactobacillus collinoides, Lactobacillus coryniformis, Lactobacillus curvatus, Levilactobacillus brevis, Lactobacillus buchneri, Lactobacillus fructivorans, Lactobacillus hilgardii, Lactobacillus fermentum, Lactobacillus reuteri, Lactobacillus ingluviei, Weissella viridescens, Bifidobacterium bifidum, Bifidobacterium adolescentis, Bifidobacterium breve, Bifidobacterium longum, Bifidobacterium animalis, Carnobacterium divergens, Corynebacterium glutamicum, Leuconostoc citreum, Leuconostoc lactis, Leuconostoc mesenteroides, Leuconostoc pseudomesenteroides, Oenococcus oeni, Pasteuria nishizawae, Pediococcus acidilactici, Pediococcus dextrinicus, Pediococcus parvulus, Pediococcus pentosaceus, Probionibacterium freudenreichii, Probionibacterium acidipropoinici, Enterococcus faecium, Enterococcus faecalis, Streptococcus thermophilus, Bacillus amyloliquefaciens, Bacillus atrophaeus, Bacillus clausii, Bacillus coagulans, Bacillus flexus, Bacillus fusiformis, Bacillus lentus, Bacillus licheniformis, Bacillus mega-terium, Bacillus mojavensis, Bacillus pumilus, Bacillus smithii, Bacillus subtilis, Bacillus vallismortis, Geobacillus stearothermophilus or mutants thereof.

In another aspect of the invention the probiotic microorganism may be selected from the genera related to the natural healthy skin microbiome including genera Lactobacillus, Lactiplantbacillus, Holzapfelia, Amylolactobacillus, Bombilactobacillus, Companilactobacillus, Lapidilactobacillus, Agrilactobacillus, Schleiferilactobacillus, Loigolactobacillus, Lacticaseibacillus, Latilactobacillus, Dellaglioa, Liquorilactobacillus, Ligilactobacillus, Furfurilactobacillus, Paucilactobacillus, Limosilactobacillus, Fructilactobacillus, Acetilactobacillus, Apilactobacillus, Levilactobacillus, Secundilactobacillus, Lentilactobacillus, Leuconostoc, Probionibacterium, Cutibacterium, Staphylococcus, Nitrosomonas, Corynebacterium, Malassezia, Aspergillus, Cryptococcus, Rhodotorula, and/or Epicoccum.

In embodiment of the present invention the probiotic strain may be Staphylococcus epidermidis, Staphylococcus hominis, Cutibacterium acnes (Probionibacterium acnes), Nitrosomonas eutropha or any combinations thereof.

In a further embodiment of the invention the probiotic strain may be a Gram-positive bacteria.

The anhydrous composition according to the present invention may comprise at least one bacterial strain selected from the group consisting of Lactiplantibacillus plantarum LB356R (DSM 33094), Lactiplantibacillus plantarum LB244R (DSM 32996), Lactobacillus crispatus LB714R (DSM 33732), Pediococcus pentosaceus LB606R (DSM 33730), Weissella viridescens LB10G (DSM 32906), Lacticaseibacillus paracasei LB113R (DSM 32907), Lacticaseibacillus paracasei LB116R (DSM 32908), Levilactobacillus brevis LB152G (DSM 32995), Lacticaseibacillus paracasei LB28R (DSM 32994), Enterococcus faecium LB276R (DSM 32997), Leuconostoc mesenteriodes LB349R (DSM 33093), Lactiplantibacillus plantarum LB316R (DSM 33091), Lactiplantibacillus plantarum LB312R (DSM 33098), Lactiplantibacillus plantarum LB679R (DSM 33731), Lactobacillus gasseri LB905R (DSM 34094), Lactobacillus crispatus LB912R (DSM 34095), Lactobacillus crispatus LB919R (DSM 34097), Lactobacillus jensenii LB918R (DSM 34096), Lacticaseibacillus paracasei LB555R (DSM 34249), Lactiplantibacillus plantarum LB681R (DSM 34250), Lactiplantibacillus plantarum LB948R (DSM 34251), Lactiplantibacillus plantarum LB958R (DSM 34252), Lactiplantibacillus plantarum LB721R (DSM 34491), Lactiplantibacillus plantarum LB760R (DSM 34492), Lactiplantibacillus plantarum LB990R (DSM 34494) Lacticaseibacillus paracasei LB857R (DSM 34493); and/or any combinations hereof and/or any mutant strains.

In an embodiment of the present invention the probiotic microorganism may be selected from the group consisting of Bifidobacterium lactis DSM10140, B. lactis LKM512, B. lactis DSM 20451, Bifidobacterium bifidum BB-225, Bifidobacterium adolescentis BB-102, Bifidobacterium breve BB-308, Bifidobacterium longum BB-536 from Zaidanhojin Nihon Bifizusukin Senta (Japan Bifidus Bacteria Center), Bifidobacterium NCIMB 41675 described in EP2823822. Bifidobacterium bifidum BB-225, Bifidobacterium adolescentis BB-102, Bifidobacterium breve BB-308, Bifidobacterium lactis HN019 (Howaru) available from International Flavors & Fragrances Inc., Bifidobacterium lactis DN 173 010 available from Groupe Danone, Bifidobacterium lactis Bb-12 available from Chr. Hansen A/S, Bifidobacterium lactis 420 Bifidobacterium breve Bb-03, B. lactis BI-04, B. lactis Bi-07, Bifidobacterium bifidum Bb-02, Bifidobacterium bifidum Bb-06, Bifidobacterium longum KC-1, Lactobacillus paracasei Lpc-37, Lactobacillus rhamnosus HN001 (Howaru), Streptococcus thermophilus 715, Streptococcus thermophilus ST21, Bifidobacterium longum 913, Lactobacillus acidophilus NCFM, Lactobacillus bulgaricus 1260 (International Flavors & Fragrances Inc.), Bifidobacterium breve M-16V (Morinaga) and/or a Lactobacillus having a probiotic effect and may be any of the following strains; Lactobacillus rhamnosus LGG (Chr. Hansen), Lactobacillus paracasei subsp. paracasei CRL431 (ATCC 55544), Lactobacillus paracasei strain F-19 from Medipharm, Inc. L. paracasei LAFTI L26 (DSM Food Specialties), Lactobacillus plantarum 299v available from Probi, L. paracasei CRL 431 (Chr. Hansen), Lactobacillus acidophilus PTA-4797, L. salivarius Ls-33 and L. curvatus 853 (International Flavors & Fragrances Inc.). Lactobacillus casei ssp. rhamnosus LC705 is described in FI Patent 92498, Valio Oy, Lactobacillus DSM15527 (Bifodan), Lactobacillus DSM15526 (Bifodan), Lactobacillus rhamnosus GG (LGG) (ATCC 53103) is described in U.S. Pat. No. 5,032,399 and Lactobacillus rhamnosus LC705 (DSM 7061), Lactobacillus reuteri strains available from BioGaia e.g. Lactobacillus reuteri ATCC PTA 6475, Propionic acid bacterium eg. Propionibacterium freudenreichii ssp. shermanii P3S (DSM 7067) described in greater details in FI Patent 92498, Valio Oy, Nitrosomonas eutropha D23 (ABIome), Staphylococcus hominis strains A9, C2, AMT2, AMT3, AMT4-C2, AMT4-GI, and/or AMT4-D12. (all from Matrisys Bioscience), L. rhamnosus PB01, L. gasseri EB01, L. curvatus EB10, L. acidophilus 5, Bifidobacterium animalis ssplactis 12, Bifidobacterium longum 536 all available from Bifodan A/S. Staphylococcus epidermidis strains M034, M038, All, AMT1, AMT5-C5, and/or AMT5-G6 (all from Matrisys Bioscience), L. plantarum YUN-V2.0 (BCCM LMG P-29456), L. pentosus YUN-V1.0 (BCCN LMG P-29455), L. rhamnosus YUN-S1.0 (BCCM LMG P-2961), Lactobacillus crispatus SJ-3C (ATCC PTA-10138), Lactobacillus crispatus CNCM I-5579 available from BASF, Lactiplantibacillus plantarum LB356R (DSM 33094), Lactiplantibacillus plantarum LB244R (DSM 32996), Weissella viridescens LB10G (DSM 32906), Lacticaseibacillus paracasei LB113R (DSM 32907), Lacticaseibacillus paracasei LB116R (DSM 32908), Levilactobacillus brevis LB152G (DSM 32995), Lacticaseibacillus paracasei LB28R (DSM 32994), Enterococcus faecium LB276R (DSM 32997), Leuconostoc mesenteriodes LB349R (DSM 33093), Lactiplantibacillus plantarum LB316R (DSM 33091), Lactiplantibacillus plantarum LB312R (DSM 33098), Pediococcus pentosaceus LB606R (DSM 33730), Lactiplantibacillus plantarum LB679R (DSM 33731), Lactobacillus crispatus LB714R (DSM 33732), Lactobacillus gasseri LB905R (DSM 34094), Lactobacillus crispatus LB912R (DSM 34095), Lactobacillus crispatus LB919R (DSM 34097), Lactobacillus jensenii LB918R (DSM 34096), Lacticaseibacillus paracasei LB555R (DSM 34249), Lactiplantibacillus plantarum LB681R (DSM 34250), Lactiplantibacillus plantarum LB948R (DSM 34251), Lactiplantibacillus plantarum LB958R (DSM 34252), Lactiplantibacillus plantarum LB721R (DSM 34491), Lactiplantibacillus plantarum LB760R (DSM 34492), Lactiplantibacillus plantarum LB990R (DSM 34494) Lacticaseibacillus paracasei LB857R (DSM 34493); and any mutant strains hereof and any combinations hereof.

The at least one viable microorganism according to the present invention may be provided in a crystal of a cryoprotectant.

The crystals comprising the microorganism may further comprise at least one further probiotic microorganism selected from the group consisting of bacteria, archaea, phages, virus, yeasts or molds or any combinations thereof.

In an embodiment of the present invention the at least one further probiotic microorganism may be a bacterium.

The use of viable probiotics for topical application is very limited and most products are based on lysates of the in-activated probiotic strain to overcome the problems of maintaining viability of the microorganisms in the topical composition. The problems observed when formulating live probiotic strains in gels, serums, emulsions, lotions and the like for topical application on the skin of mammals may include lack of viability and stability.

Compositions for topical applications are typically to be stable for months at room temperature, this is a major problem for maintaining viability of live probiotic microorganisms in skin care products.

The present invention solves the problem of stabilizing the live microorganisms, in particular the probiotic strains, in a composition for topical use on skin or mucous membranes.

The anhydrous substance disclosed herein remain essentially water free and contain no more than a trace of water. The invention relates to anhydrous topical compositions comprising only anhydrous ingredients and no added water. Examples of such compositions based on lipids, oils and fats. Such anhydrous composition is directly applicable as a cosmetic or pharmaceutical formulation.

The anhydrous substance may comprise a vegetable lipid.

Furthermore, the anhydrous substance may comprise a vegetable oil and/or a vegetable fat and/or a hydrogenated oil and/or a vegetable wax and/or a fatty pasty compound (in particular a vegetable fatty pasty compound), or any combination hereof.

In an embodiment of the present invention, the anhydrous topical composition comprises less than 10% (w/w) water relative to the anhydrous topical composition, such as less than 8% (w/w) water, e.g. less than 6% (w/w) water, such as less than 4% (w/w) water, e.g. less than 2% (w/w) water, such as less than 1.5% (w/w) water, e.g. less than 1% (w/w) water, such as less than 0.5% (w/w) water.

Preferably, the anhydrous composition comprises no added water. Some anhydrous substances, e.g. anhydrous vegetable oils, may comprise traces of water and may be essentially water free. More preferable, the anhydrous substances (or other anhydrous ingredients) used in the anhydrous composition may comprise less than 3% water, more preferable the anhydrous composition may comprise less than 1% water, more preferable the anhydrous composition may comprise less than 0.5% water, more preferable the anhydrous composition may comprise less than 0.1% water, more preferable the anhydrous composition may comprise less than 0.05% water.

In one embodiment of the invention the anhydrous composition may be an oil.

Preferably, the “oil” according to the present invention may relate to an oil being liquid at storage temperature, thus the liquid oil has a freezing point below 25 °C. The freezing point of the oil may relate to the temperature at which the oil may change between liquid phase and solid phase.

Preferably, the oil may be a vegetable oil which can be absorbed by the skin or the mucous membrane.

In an embodiment of the invention the oil may be a vegetable oil selected from the group consisting of almond oil, sunflower oil, hemp oil, CBD oil, Cannabis oil, Evening prim rose, Borage oil, baobab oil, acai oil, Almond sweet oil, Rose Hip oil, jojoba oil, Jojoba Golden oil, Camomile oil, shea oil, coconut oil, calendula oil, sea buck-thorn oil, Jafflower oil, castor oil, olive oil, corn oil, soya bean oil, cotton seed oil, wheat oil, linseed oil, apricot kernel oil, argan oil, camelina oil, comfrey oil, grape seed oil, kiwi seed oil, mullein oil, rapeseed oil, peach kernel oil, thistle oil, sesame oil, and a combination hereof.

In an embodiment of the present invention, the anhydrous composition may also comprises an oil selected from the group consisting of acai, acai berry, almond, almond sweet, aloes vera, andiroba, apricot kernel, Arnica, argan, avocado, babassu, baobab, black berry seed, black cumin, black currant seed, blueberry, borage, brazil nut, broccoli seed, buriti, calendula, Camellia seed, Cannabis oil including CBD and THC, canola, copaiba balsam, cape chestnut (yangu), carrot (Daucus carrota), castor, chardonnay grape, chaulmoogra, cherry Kernel, chia seed, chickweed, coconut, coconut fractionated, cotton seed, comfrey, corn, Crambe seed, cranberry seed, cucumber seed, echium seed, evening primrose, emu, flax seed, grape seed, hazelnut, hemp seed, horsechest nut seed, jojoba, karanj seed, kiwi seed, kukuinut, macadamia nut, marula, marshmallow, manketti, meadowfoam, milk thistle seed, moringa, mullein, mustard seed, neem, olive, palm, Papaya seed, passionflower seed, peach kernel, peanut, Perilla, pomegranate, Pentaclethra macroloba, pumpkin seed, rape seed, raspberry seed, rice bran, rosehip, St. John's Wort oil, safflower, sea buckthorn pulp, sheabutter oil, sesame roast-ed, sesame seed, soya been, sunflower, tamanu (Calophyllum In-ophyllum), thistle, tomato, turkey red, sangre de drago, walnut, watermelon seed, wheatgerm, Abyssinian, Colza, bees wax, lanolin, linseed, Mortierella oil, ongokea, paraffinum liquid, peacan, Pegui, Poppy seed, Pracaxi, rapeseed, soybean, tall, tung, veronica, Wheat germ, yangu seed and any combination thereof.

In an embodiment of the present invention, the anhydrous composition may comprise an oil selected from sunflower oil, jojoba oil, olive oil, rapeseed oil and/or almond oil or any combinations hereof.

Anhydrous ingredients which may suitably be used in the anhydrous composition according to the invention may include waxes, such as beeswax, paraffin wax, animal stearates, glycol esters, mono and diglycerides and wax mixtures; oils, such as olive oil, sunflower oil, canola oil, coconut oil, corn oil, palm oil, sesame oil, peanut oil, along with medium chain triglycerides; an emulsifier, for example a phospholipid such as lecithin; a glyceride such as a mono- or di-glyceride or a combination thereof, for example a stearate, for example glycerol monostearate, such as an animal or vegetable stearate, and/or a palmitate such as glycerol monopalmitate, a dispersant or a surfactant such as polyethylene glycols.

The anhydrous composition comprises anhydrous ingredients including lipids, oils, fats, waxes and water-free organogelators. Structuring the anhydrous composition as a formulation suitable for topical applications can be done by using a water-free organogelator and be used to create a partly liquid two-phase water-free system (oil-gel).

An organogel, also called oleogel, may be a class of gel made of oils, fats and waxes in a three-dimensional network formed by an organogelator.

Many types of anhydrous organogelators have been developed, including vegetable waxes and hydrogenated vegetable oils such as Rapeseed wax (hydrogenated rapeseed oil), candelilla wax (Euphorbia cerifera cera), rice bran wax (Oryza Sativa Bran Cera), berry wax (Rhus verniciflua peel cera/Rhus succedanea fruit cera), Oliwax (hydrogenated olive oil), Tea wax (Camellia sinensis cera), Myrica fruit wax (Myrica cerifera fruit wax), sunflower wax (Hydrolyzed sunflower seed wax), Sunflower seed wax (Helianthus Annuus Seed cera, ascorbyl palmitate, tocopherol), Castor wax (Hydrogenated castor oil), carnauba wax (Copernicia cerifera cera), or any other vegetable based wax (hydrogenated vegetable oil). Non vegan natural waxes which can be used as organogelator is bee's wax.

When used as organogelators the waxes can be mixed to create a topical formulation with particular physical properties.

The composition according to the invention can comprise at least one solid compound (solid at ambient temperature and atmospheric pressure) selected more particularly from waxes, pasty compounds and mixtures thereof.

“Wax”, within the meaning of the invention, designates a lipophilic compound solid at ambient temperature (25 °C), having a melting temperature greater than or equal to 40 °C and less than or equal to 120 °C, more particularly less than or equal to 90 °C.

Within the meaning of the invention, the melting temperature (or melting point) is the temperature of the most endothermic peak observed in thermal analysis (DSC), as described in ISO 11357-3: 1999.

The melting point of a solid fat can be measured using a differential scanning calorimeter (DSC), for example the calorimeter sold under the trade name “DSC 2000” by TA Instruments with the “TA Universal Analysis” software.

The measurement protocol is as follows:

A 5 mg sample of wax may be placed in a crucible and subjected to a first temperature rise from −20 °C to 120 °C, at a heating rate of 10 °C/minute, and is then cooled from 120 °C to −20 °C at a cooling rate of 10 °C/minute and finally subjected to a second temperature rise from −20 °C to 120 °C at a heating rate of 5 °C/minute. During the second temperature rise, the melting point of the solid fat is measured, corresponding to the temperature of the most endothermic peak of the melting curve observed.

More particularly, the wax can be selected from polar waxes in particular alcohol waxes; esters such as waxes of animal or plant origin, such as beeswax, lanolin, sunflower, candelilla, carnauba, rice bran, montan, hydrogenated jojoba oil; waxes resulting from the hydrogenation of esters obtained from C6-C22 fatty alcohols of plant origin and vegetable oil; waxes having the formula R₁COOR₂ wherein R₁ and R₂ are aliphatic linear, branched or cyclic chains wherein the number of atoms varies from 10 to 50, that can contain a heteroatom, in particular oxygen, and the melting temperature of which varies from 40 to 120 °C; partial or total, preferably total, esters of a C₁₆-C₃₀ carboxylic acid, saturated, with glycerol, such as glyceryl tristearate or tribehenate, and mixtures thereof.

Mention can also be made of non-polar hydrocarbon waxes (in other words consisting solely of carbon and hydrogen atoms), resulting from the transformation of petroleum, such as for example polyethylene waxes, polymethylene waxes (synthetic wax, Fischer-Tropsch waxes), alone or in mixtures.

Preferably the composition comprises 10 to 40% (w/w) of at least one wax by weight of the total weight of the composition, preferably 12 to 30% (w/w) by weight.

“Pasty compound”, within the meaning of the present invention, means a lipophilic compound, which at a temperature of 25 °C has a liquid fraction and a solid fraction. Thus, a pasty compound can have a commencing melting point of less than 25 °C.

The melting point of the fatty pasty compound may be determined according to the same principle as that detailed previously for waxes. In the case of a pasty compound (or pasty fat), the measurement protocol is however as follows:

A 5 mg sample of pasty fat placed in a crucible may be subjected to a first temperature rise from −20 °C to 100 °C, at a heating rate of 10 °C/minute, and is then cooled from 100 °C to −20 °C at a cooling rate of 10 °C/minute and finally subjected to a second temperature rise from −20 °C to 100 °C at a heating rate of 5 °C/minute. The melting point of the pasty fat is the value of the temperature equivalent to the top point of the peak of the curve representing the variation in the difference in power absorbed as a function of temperature.

It should be noted that the liquid fraction by weight of the pasty fat at ambient temperature is equal to the ratio of the enthalpy of fusion consumed at ambient temperature to the enthalpy of fusion of the pasty fat. The enthalpy of fusion of the pasty fat is the enthalpy consumed by the latter to change from the solid state to the liquid state. The pasty fat is said to be in the solid state when the entire mass thereof is in solid crystalline form. The pasty fat is said to be in the liquid state when the entire mass thereof is in liquid form. The enthalpy of fusion of the pasty fat is the quantity of energy required to change the pasty fat from the solid state to the liquid state. It is expressed in J/g. The enthalpy of fusion of the pasty fat is equal to the area under the curve of the thermogram obtained.

Preferably, this or these pasty hydrocarbon compounds may be selected from vaseline; plant butters, such as in particular mango, shea, cupuacu, murumuru, cocoa, babassu and jojoba butters; completely or partially hydrogenated vegetable oils, such as for example hydrogenated soya oil, hydrogenated copra oil, hydrogenated rapeseed oil, mixtures of hydrogenated vegetable oils such as products with the INCI name hydrogenated vegetable oil, partially hydrogenated olive oil; esters of hydrogenated castor oil and of C₁₆-C₂₂ fatty acids, bis-diglyceryl polyacyladipate-2; products with the INCI name Hydrogenated Coco-Glycerides; esters of dilinoleic alcohol dimer and/or of dilinoleic acid such as for example products with the following INCI names: Bis-Behenyl/Isostearyl/Phytosteryl Dimer Dilinoleyl Dimer Dilinoleate, Phytosteryl/Isostearyl/Cetyl/Stearyl/Behenyl Dimer Dilinoleate), Stearyl stearate, Hydrogenated Castor Oil Dimer Dilinoleate, and mixtures thereof.

Preferably the composition comprises 10 to 50% (w/w) of at least one pasty fatty compound by weight of the total weight of the composition, preferably 20 to 40% by weight.

Organogelator may be used in concentrations up to 40% (w/w) of the anhydrous substance. More preferable the concentration of the organogelator may be less than 20% (w/w) and even more preferable the concentration may be less than 17% (w/w).

In an embodiment of the present invention the organogelator in the anhydrous composition may be a hydrogenated vegetable oil. In a preferred embodiment, the anhydrous organogelator may be hydrogenated olive oil, hydrogenated rapeseed oil or hydrogenated sunflower oil.

Water free polymers and synthetic waxes can also be used to gel the anhydrous substance.

The polymer suited for the present invention may be hydrogenated oils and polyurethane polymers and co-polymers being able to gel oils. Examples of such polyurethane polymers are disclosed in WO18185432.

Preferably the polyurethane polymers may be selected from Oilkemia 5S polymer from Lubrisol and EstoGel M polymer from Polymerexpert or the like. Preferably, the polyurethane polymers according to the present invention comprises caprylic/capric triglycerides (e.g. castor oil) and may typically be co-polymers of castor oil and polyurethane.

The polymer of the invention may be a polyurethane based on vegetable oils. Some vegetable oils used for production of polyurethane may need chemical modifications before polymerization.

In a preferred embodiment of the present invention the polyurethane polymer may be based on Castor oil.

In an embodiment of the present invention the polymer may comprise more than 10% w/w castor oil, in a more preferred embodiment of the present invention the polyurethane polymer may comprise more than 15% w/w castor oil, an even more preferred embodiment of the present invention the polyurethane polymer may comprise more than 20% w/w castor oil, an even more preferred embodiment of the present invention the polyurethane polymer may comprise more than 20% w/w castor oil.

The anhydrous substance may comprise a polyurethane polymer obtained from (based on) caster oil. In an embodiment of the present invention the anhydrous substance may comprise more than 10% (w/w) polyurethane polymer obtained from (based on) caster oil, such as more than 15% (w/w) polyurethane polymer obtained from (based on) caster oil, e.g. more than 15% (w/w) polyurethane polymer obtained from (based on) caster oil, such as more than 20% (w/w) polyurethane polymer obtained from (based on) caster oil, e.g. more than 25% (w/w) polyurethane polymer obtained from (based on) caster oil, such as more than 30% (w/w) polyurethane polymer obtained from (based on) caster oil, e.g. more than 35% (w/w) polyurethane polymer obtained from (based on) caster oil, such as more than 40% (w/w) polyurethane polymer obtained from (based on) caster oil, e.g. more than 45% (w/w) polyurethane polymer obtained from (based on) caster oil, such as more than 50% (w/w) polyurethane polymer obtained from (based on) caster oil.

The invention may not be limited to commercially available water free polyurethane products but to any water free polyurethane polymer/co-polymer product being able to gel anhydrous carriers.

The caprylic/capric triglyceride and polyurethane polymers may be used in the oil in a concentration from 0.1% (w/w) to 20% (w/w). Preferable in the concentration from 0.3% (w/w) to 10% (w/w) and more preferable from 0.5% (w/w) to 6% (w/w).

Synthetic anhydrous waxes may include microcrystalline wax which may be produced by de-oiling petrolatum as part of its refining process. Paraffin wax may be derived from petroleum. Ozokerite, ceresin, and montan waxes may originally be mineral waxes which are derived from coal and shale. Ozokerite for cosmetics may also be synthesized from petroleum, exactly like microcrystalline waxes.

It should be noted that embodiments and features described in the context of one of the aspects of the present invention also apply to the other aspects of the invention.

It will be further understood that, unless the context dictates otherwise, the embodiments described herein may be combined.

When used herein, the term topical may relate to formulations that are adapted for application to body surfaces (e.g. the skin or mucous membranes). Mucous membranes may include the mucosa of the vagina, the penis, the urethra, the bladder, the anus, the mouth, the nose and/or the ear.

The present invention relates to new methodologies and compositions for stabilization of live probiotic strains in a composition for topical use to skin and/or mucous membranes.

The utilization of these compositions comprising probiotic bacteria further facilitate the probiotic effects on skin of both humans and animals.

The present invention relates to methodologies for preparing an anhydrous composition comprising an anhydrous substance, at least one viable microorganism and a deliquescent substance, preferably a deliquescent salt.

Furthermore, the present invention provides a topical therapeutic composition for the treatment or prevention of a skin disorder.

Preferably, the present invention provides a topical therapeutic anhydrous composition for the treatment or prevention of a skin disorder, comprising a therapeutically-effective concentration of one or more viable microorganisms, e.g. live probiotic species or probiotic strains or live biotherapeutic products (LBP), within a pharmaceutically-acceptable anhydrous substance suitable for topical administration on the skin or mucous membranes of a mammal. Preferably, the probiotic strain possesses the ability to maintain viable in the anhydrous composition at room temperature.

In another aspect, the invention relates to an anhydrous composition according to the present invention further comprising a cosmetically acceptable anhydrous ingredient. Preferable the anhydrous composition may be provided in an anhydrous liquid, anhydrous fluid, or anhydrous ointment form for topical application.

The anhydrous composition may advantageously further comprise other probiotics, prebiotics, or other active substances and/or may preferably also contain one or more of the following substances selected from antioxidants, vitamins, coenzymes, fatty acids, amino acids, and cofactors.

In a preferred embodiment the antioxidant may be Vitamin E or a variant thereof (wherein the variants may be selected from alfa, beta, gamma, delta tocopherol, tocotrienols and tocomonoenols).

Preferably, the antioxidant is present at a concentration below 5% (w/w) of the anhydrous composition, preferably below 4% (w/w), preferably below 3% (w/w), preferably in the range of 0.01-5% (w/w), preferably in the range of 0.02-4% (w/w), preferably in the range of 0.03-3% (w/w), preferably in the range of 0.06 to 1% (w/w).

Preferably, the antioxidant may be tocopherol.

In a further embodiment the tocopherol may be provided in the anhydrous substance, or in the anhydrous composition. Preferably, tocopherol may represent a concentration below 5% (w/w) of the anhydrous substance or composition, such as a concentration below 4% (w/w), e.g. a concentration below 3% (w/w), such as in the range of 0.01-5% (w/w), e.g. in the range of 0.1-4% (w/w), such as in the range of 0.5-3% (w/w), preferably in the range of 0.06 to 1% (w/w) or in the range of 1-2% (w/w).

Vegetable oils may contain natural antioxidants, in an embodiment of the invention further antioxidants may be incorporated into the composition. Antioxidants preferably added to a vegetable oil may be tocopherol (added in a range between 0.25 to 2.5% (w/w)) and/or Rosemary extract (added in a range between 0.1 to 0.75% (w/w)).

The composition according to the invention preferably comprises, by weight of the total weight of the composition:
50% to 99.5% by weight, preferably 60% to 99% by weight, of at least one anhydrous substance, preferably chosen from vegetable oils, vegetable waxes, vegetable pasty fatty compounds and their mixtures;
0.001% to 20% by weight, preferably 0.005% to 10% by weight, preferably 0.01% to 5% by weight, of at least one viable microorganism;
0.2% to 2% by weight, preferably 0.2% to 1% by weight of at least one deliquescent substance; and
optionally 0.01% to 2% by weight, preferably 0.02% to 1% by weight of at least one antioxidant.

“Viability” of microorganisms may be measured as Colony Forming Units CFU. A “decrease” in viability of microorganisms may be determined as the difference in CFU/g or CFU/ml as compared to the CFU/g or CFU/ml at the time of formulating the composition.

A “decrease” in viability may be “statistically significant” as compared to the viability determined at the time of formulating the composition. Decrease may be measured as a log reduction and may include a log reduction of 0.1 or more, such as of 0.5 or more, e.g. of 1.0 or more, such as of 1.5 or more, e.g. of 2.0 or more, such as of 2.5 or more, e.g. of 3.0 or more, such as of 3.5 or more, e.g. of 4.0 or more, such as of 4.5 or more, e.g. of 5.0 or more.

The microorganisms according to the invention may be provided in isolated or purified form. The term “isolated” may mean that the microorganism is cultivated as a monoculture and may be derived from the culture medium including their natural medium. The term “purified” is not restricted to absolute purity.

The microorganisms may advantageously be present in viable dried form. The dried viable microorganism may be spray-dried or lyophilized or vacuum dried.

In an embodiment of the invention the probiotic strain may be used as a live isolated microorganism in a dried form. Preferably, the at least one viable microorganism may be a lyophilized microorganism, preferably a lyophilized microorganism comprising a cryoprotective agent.

In a preferred embodiment of the invention the probiotic strain may be used as a viable isolated probiotic strain dried into a crystal of cryoprotectant. The crystal may comprise at least 2% cryoprotectant, such as at least 5% cryoprotectant, e.g. at least 10% cryoprotectant, such as at least 15% cryoprotectant.

The cryoprotectant may be selected from maltodextrin, trehalose, saccharose, lactose, mannitol, sucrose, glycerol, sorbitol, dextran, inulin, citrate, ascorbate, calcium chloride or a combination hereof.

In addition, it is preferable for the dried microorganism to be present in the anhydrous composition in an amount by weight of 0.001% (w/w) to 20% (w/w), preferably 0.005% to 10% (w/w), especially preferably 0.01% to 5% (w/w).

The anhydrous composition according to the present invention may be formulated for the administration of from approximately 1×10² to 1×10¹⁴ CFU of viable bacteria per gram anhydrous composition, more preferably from approximately 1×10³ to 1×10¹⁰ CFU/g, and most preferably from approximately 1×10⁴ to 1×10⁹ CFU of viable bacteria per gram of anhydrous composition.

The anhydrous composition according to the present invention, wherein the at least one microorganism may be present in the anhydrous composition in a concentration above 104 CFU per gram anhydrous composition, such as above 105 CFU per gram anhydrous composition, e.g. above 10⁶ CFU per gram anhydrous composition, such as above 107 CFU per gram anhydrous composition, such as above 10⁸ CFU per gram anhydrous composition.

In an embodiment of the invention the dosage of live probiotic microorganisms in the anhydrous composition may be above approximately 1×10³ CFU of viable bacteria per gram of the anhydrous composition, preferably above approximately 1×10⁴ CFU/g, even more preferably above approximately 1×10⁵ CFU/g, even more preferably above approximately 1×10⁶ CFU/g.

In the present context the term “effective” depends upon the context in which it is being applied. In the context of administering a composition comprising a viable microorganism topically on a skin or mucous membrane surface, an effective amount may be the number of viable microorganisms determined as CFU/gram which may have a probiotic effect on skin or mucous membranes.

The anhydrous topical compositions according to the present invention surprisingly showed an increased stability on the microbial viability when providing an anhydrous composition comprising an anhydrous substance, at least one viable microorganism and a deliquescent substance, preferably an inorganic salt.

In one embodiment of the present invention the increased stability may be measured as a log reduction in CFU/g per month of storage at 25 degrees Celsius.

In an embodiment of the present invention the increased stability may be a log reduction in CFU/g of less than 0.3 log per month of storage at 25 degrees Celsius, more preferably a log reduction in CFU/g of 0.2 log per month of storage at 25 degrees Celsius, more preferably a log reduction in CFU/g of 0.1 log per month of storage at 25 degrees Celsius. Crystals comprising microorganisms may be less than 2 mm in diameter, more preferable less than 1 mm in diameter, typically in the interval from 5 μm to 1 mm in diameter. More preferable in the interval 10 μm to 0.5 mm, such as in the range of 50-250 μm, e.g. in the range of 100-150 μm.

The crystal size may be determined by sieving using predefined mesh sizes.

In an embodiment of the present invention the at least one microorganism may be a single microorganism or a mixture of microorganisms, such as a single bacterium or a mixture of bacterial strains.

A “mammal” include, but are not limited to, humans, primates, farm animals, sport animals, rodents, and pets. Non-limiting examples of non-human mammal subjects may include rodents such as hamsters, and guinea pigs; rabbits; dogs; cats; sheep; pigs; piglets; sows; poultry; turkeys; broilers; minks; goats; cattle; horses; and non-human primates such as apes and monkeys.

In an embodiment of the present invention the anhydrous composition may be used for topical application to human skin.

In an embodiment of the present invention the anhydrous composition may be used for topical application to human mucous membranes.

In an embodiment of the present invention the anhydrous composition may be used for application to mucous membranes of the genitals.

In an embodiment of the present invention the anhydrous composition may be used for topical cosmetic applications.

In an embodiment of the present invention the anhydrous composition may be used for topical pharmaceutical applications.

In an embodiment of the present invention the anhydrous composition may be used for a cosmetic, a prophylaxis medicament or a medicament for the treatment of a disease, dysfunction, or disorder.

In an embodiment of the present invention the anhydrous composition may be used for a cosmetic for anti-age treatment.

In one aspect of the invention the anhydrous composition comprising the microorganism further comprises a prebiotic. “Prebiotics” are components that increase the growth of specific microorganisms. “Synbiotics” are compositions comprising at least one probiotic and at least one prebiotic. Such compositions may be understood to encourage the growth of beneficial microorganisms (e.g. the probiotic). Thus, powerful synbiotics may be based on a combination of specific strains of probiotic microorganisms with carefully selected prebiotics. They can lead to an important health benefit to a mammal.

According to another aspect of the present invention there is provided a probiotic composition comprising the probiotic microorganism and at least one more active ingredient, like a prebiotic. Prebiotics may refer to chemical products that induce the growth and/or activity of commensal microorganisms of the microbiota (e.g., bacteria and fungi) that contribute to the well-being of their host. Prebiotics stimulate the growth and/or activity of advantageous bacteria that colonize the skin.

Prebiotics may include one or more oligosaccharides. Some oligosaccharides that may be used as prebiotics are fructooligosaccharides (FOS), xylooligosaccharides (XOS), polydextrose, pectins, galactooligosaccharides (GOS) or human milk oligo saccharides (HMO). Moreover, disaccharides like lactulose, lactose or some monosaccharides such as or tagatose may also be used as prebiotics.

The other active ingredient (or other ingredients) may not be limited in any way. In an embodiment of the present invention at least one prebiotic compound may be added to the anhydrous composition of the invention, i.e. as other ingredient.

Prebiotics according to the present invention may include all those compounds which can be metabolized by probiotics. Prebiotics can thus serve as a food source for probiotics.

Prebiotics are well known in the art and when used in the present invention there is no particular limitation of the prebiotic as such.

In an embodiment of the present invention the at least one prebiotic in the anhydrous composition may be selected from the following group of compounds and compositions carbohydrates, glucans, alpha-glucans, beta-glucans, mannan-oligosaccharides, inulin, oligofructose, human milk oligosaccharides (HMO), galactooligosaccharides (GOS), lactulose, lactosucrose, galactotriose, fructooligosaccaride (FOS), cellobiose, cellodextrins, cylodextrins, maltitol, lactitol, glycosilsucrose, betaine, Vitamin E or a variant thereof (wherein the variants are selected from alfa, beta, gamma, delta tocoferols, tocotrienols and tocomonoenols). Optionally, mannanoligosaccharides and/or inulin may be preferred.

HMOs may include lacto-N-tetraose, lacto-N-fucopentaose, lacto-N-triose, 3′-sialyllactose, lacto-N-neofucopentaose, sialic acid, L-fucose, 2-fucosyllactose, 6′-sialyllactose, lacto-N-neotetraose, 3-fucosyllactose, or any combination hereof.

Preferably, at least one of the following prebiotic compounds may be used in the topical anhydrous composition of the present invention lactose, beta-glucans, mannan-oligosaccharides, inulin, oli-gofructose, galactooligosaccharides (GOS), lactulose, lactose, lactosucrose, galactotriose, fructo-oligosaccaride (FOS), cellobiose, cellodextrins, cylodextrins, maltitol, lactitol, glycosilsucrose, betaine, lacto-N-tetraose, lacto-N-fucopentaose, lac-to-N-triose, 3′-sialyllactose, lacto-N-neofucopentaose, sialic acid, 2-fucosyllactose, 6′-sialyllactose, lacto-N-neotetraose, 3-fucosyllactose or a combination hereof. Optionally, lactose and/or mannan-oligosaccharides and/or inulin may be preferred.

Fucose, in particular L-fucose, may be believed to strengthen natural defence of skin, stimulate epidermis immune defence and/or prevent and/or treat cutaneous autoimmune disease. In one preferred embodiment of the invention the anhydrous composition comprises L-fucose and/or D-fucose.

In an embodiment of the present invention the anhydrous composition further comprises L-fucose and/or D-fucose in a concentration in the anhydrous composition in the range of 10 mM to 500 mM, such as in the range of 50-400 mM, e.g. in the range of 100-300 mM, e.g. in the range of 150-250 mM.

The present invention provides several advantages. In particular, viability of the microorganisms is kept in the composition even at storage at room temperature (about °C). The microorganisms may be activated by the temperature and moisture of the skin releasing the microorganisms from the anhydrous composition as it is absorbed by the skin.

All patent and non-patent references cited in the present application, are hereby incorporated by reference in their entirety.

The present invention successfully addresses the shortcomings of the presently known compositions for topical use. Known compositions for topical use are not able to maintain the viability of the microorganisms at room temperature for long term storage.

A preferred embodiment of the present invention relates to a deliquescent substance, such as a deliquescent salt, to stabilize viable microorganisms in an anhydrous topical composition.

Provided is also a procedure to produce an anhydrous composition comprising a viable microorganism and a deliquescent substance, e.g. a water soluble deliquescent salt for topical use.

A preferred embodiment of the present invention relates to a process for producing an anhydrous composition, the method comprises the steps of:
Providing an anhydrous substance;
Mix the anhydrous substance with at least one viable microorganism; and
mix the anhydrous substance with a deliquescent substance;
providing the anhydrous composition.

In an embodiment of the present invention:
the anhydrous substance may be mixed and homogenized with the microorganism before the deliquescent substance is added;
the anhydrous substance is mixed and homogenized with the deliquescent substance before the microorganism is added; or
the anhydrous substance may be mixed with the microorganism and the deliquescent substance before being homogenized,
   providing the anhydrous product.

A preferred embodiment of the present invention relates to a process for producing an anhydrous composition, the method comprises the steps of:
Providing an anhydrous substance;
Add a deliquescent substance, like a deliquescent salt to the anhydrous substance;
Homogenize the mixture of anhydrous substance and deliquescent substance;
Optionally, cool the melted anhydrous substance comprising the deliquescent substance, like a deliquescent salt, to a temperature below °C:
Add a at least one probiotic microorganism; and
homogenize the mixture providing the anhydrous composition.

A Further preferred embodiment relates to a method for providing a topical anhydrous composition comprising an anhydrous substance, at least one viable microorganism and a deliquescent substance, such as a deliquescent salt, wherein the deliquescent substance may be subjected to a step of dehydration or partly dehydration before being mixed into the anhydrous substance providing the anhydrous composition.

The anhydrous substance or the mixture of the anhydrous substance and the deliquescent substance may be cooled to a temperature below 40 °C before the at least one microorganism may be added, such as below 35 °C, e.g. a temperature below 30 °C, such as to a temperature below 25 °C, e.g. a temperature below 20 °C, such as in the range of 10-40 °C, e.g. in the range of 15-35 °C, such as in the range of 18-30 °C, e.g. in the range of 20-25 °C.

The deliquescent substance, e.g. the deliquescent salts, may be dried to remove water/moisture from the deliquescent substance. The drying of the deliquescent substance may be provided by heating at a temperature of 100-200 °C, such as a temperature of 125-175 °C, e.g. for about 150 °C for a period of at least 30 minutes, such as for at least 1 hour, e.g. for at least 1½ hours, such as for at least 2 hours, e.g. for at least 3 hours, such as for at least 4 hours, e.g. for at least 5 hours, such as for at least 6 hours, e.g. for a period of 10 minutes to 10 hours, such as for a period of 30 minutes to 8 hours, e.g. for a period of 1-6 hours, such as for 2-5 hours, e.g. for 3-4 hours, before being added to the anhydrous substance.

The drying of the deliquescent substance, e.g. the deliquescent salts, may be done in a Binder Model ED 23 drying chamber.

In an embodiment of the present invention the anhydrous composition according to the present invention may not comprise a coated or a microencapsulated microorganism, and/or a coated or a microencapsulated deliquescent substance.

Coating and microencapsulation may relate to tiny particles or droplets of a material (the core material) which are surrounded by a protective shell, forming small capsules. This shell acts as a barrier between the core material and its environment. The shell material is typically a polymeric substance.

In a preferred embodiment of the present invention the anhydrous composition comprises a mixture of the microorganisms, the deliquescent substance, and the anhydrous substance.

A mixture may be a combination of two or more substances (the anhydrous substance and the deliquescent substances and the microorganisms) in which each substance may retains its own chemical properties. Mixtures may be homogeneous (uniformly mixed) or heterogeneous (non-uniformly mixed).

In the mixture according to the present invention the deliquescent substances, the microorganisms, and the anhydrous substance may form a homogenous or a heterogeneous mixture in which the deliquescent substances and/or the microorganisms may be dispersed within the anhydrous substance.

The anhydrous composition according to the present invention may be provided according to the one of the following procedures.

### Procedure 1:

Step 1; Melt the anhydrous substance, like fats and mix with all anhydrous ingredients;
Step 2; Cool the melted anhydrous substance (and anhydrous ingredients) to room temperature (approximately 20 °C) and add at least one probiotic microorganism;
Step 3; Homogenize the mixture of anhydrous substance (and anhydrous ingredients) and at least one probiotic microorganism;
Step 4; Add a deliquescent substance, like a deliquescent salt,
Step 5; homogenize the mixture providing the anhydrous composition.

### Procedure 2:

Step 1; Melt the anhydrous substance, like fats and mix with all anhydrous ingredients
Step 2; Add a deliquescent substance, like a deliquescent salt to the melted anhydrous substance (and anhydrous ingredients)
Step 3; Homogenize the mixture of anhydrous substance (and anhydrous ingredients) and deliquescent substance;
Step 4; Cool the melted anhydrous substance (and anhydrous ingredients) to room temperature (approximately 20 °C) comprising the deliquescent substance, like a deliquescent salt:
Step 5; Add at least one probiotic microorganism;
Step 6; homogenize the mixture providing the anhydrous composition.

### Procedure 3:

Step 1; Melt the anhydrous substance, like fats or waxes and mix with all anhydrous ingredients
Step 2; Cool the melted anhydrous substance (and anhydrous ingredients) to room temperature (approximately 20 °C) and add a deliquescent substance, like a deliquescent salt and the at least one probiotic microorganism;
Step 3; homogenize the mixture providing the anhydrous composition.

The invention will now be described in further details in the following non-limiting examples.

### EXAMPLES

### Example 1

Anhydrous compositions were produced following the procedure:
Step 1; Melt fat and waxes solid at room temperature
Step 2; Add liquid oil and other anhydrous ingredients e.g. Vitamin E and homogenize
Step 3: Add dried deliquescent salt and homogenize.
Step 4; Cool to a temperature of 20-30 °C and add freeze-dried probiotic microorganism and homogenize.

The following compositions were produced following the procedure above.

### Composition 1:

Shea butter fat: 10 g
Almond oil: 50 g
Jojoba oil: 38.5 g
MgCl₂: 0.5 g (Sigma 208337)
CaCl₂: 0.5 g (Sigma C3306)
Freeze dried microorganisms: Lactiplantibacillus plantarum LB244R® (DSM 32996): 0.5 g (correspond to approximately 107 CFU/ml of final composition)

The deliquescent salts were dried to remove water by heating at 150 °C in Binder Model ED 23 drying chamber for at least 1 hour before step 3.

### Composition 2:

Shea butter fat: 20 g
Almond oil: 50 g
Hydrogenated olive oil (wax): 9 g
Sunflower oil: 18 g
Tocopherol: 1 g
MgCl₂: 1 g
Freeze dried microorganisms: Lactiplantibacillus plantarum LB356R® (DSM 33094): 1 g (correspond to approximately 10⁸ CFU/ml of final composition)

The deliquescent salts were dried to remove water by heating at 150 °C in Binder Model ED 23 drying chamber for at least 1 hour before step 3.

### Composition 3:

Cocoa butter fat: 80 g
Sunflower oil: 11.9 g
Hydrogenated olive oil wax: 6 g
Tocopherol: 0.1 g
MgCl₂: 2 g
Freeze dried microorganisms: Pediococcus pentosaceus LB606R (DSM 33730) 0.5 g and Lactiplantibacillus plantarum LB356R® (DSM 33094) 0.5 g (correspond to approximately 10⁹ CFU/ml of final composition)

The deliquescent salts were dried to remove water by heating at 150 °C in Binder Model ED 23 drying chamber for at least 1 hour before step 3.

### Composition 4:

Jojoba oil: 72 g
Almond oil: 10 g
Hydrogenated olive oil: 15 g
Tocopherol: 1 g
MgCl₂: 1 g
Freeze dried microorganisms: Pediococcus pentosaceus LB606R (DSM 33730) 1 g (correspond to approximately 109 CFU/ml of final composition)

The deliquescent salts were dried to remove water by heating at 150 °C in Binder Model ED 23 drying chamber for at least 1 hour before step 3.

### Composition 5:

Jojoba oil: 70 g
Almond oil: 28 g
Tocopherol: 0.5 g
MgCl₂: 0.5 g
Freeze dried microorganisms: Lactiplantibacillus plantarum LB244R® 1 g (correspond to approximately 109 CFU/ml of final composition)

The deliquescent salts were dried to remove water by heating at 140 °C in Binder Model ED 23 drying chamber for at least 1 hour before step 3.

### Composition 6:

Vegetable beeswax: 38.5 g
Sunflower oil: 20 g
Rapseed oil: 20 g
Jojoba oil: 20 g
Tocopherol: 0.5 g
MgSO₄: 0.35 g (Sigma 434183)
Freeze dried microorganisms: Lactiplantibacillus plantarum LB714R® (DSM 33732) 1 g (correspond to approximately 5*10⁸ CFU/ml of final composition)

### Composition 7:

Shea butter fat: 10.5 g
Almond oil: 50 g
Jojoba oil: 38.5 g
CaCl₂: 0.5 g (Sigma C3306)
Freeze dried microorganisms: Lactiplantibacillus plantarum LB244R® (DSM 32996): 0.5 g (correspond to approximately 10⁸ CFU/ml of final composition)

The deliquescent salts were dried to remove water by heating at 150 °C in Binder Model ED 23 drying chamber for at least 1 hour before step 3.

The above description is for the purpose of teaching the person of ordinary skill in the art how to utilize the disclosure provided herein. It is not intended to detail all of those obvious modifications and variations which will become apparent to the skilled worker upon reading the description of the present invention. It is intended, however, that all such obvious modifications and variations be included within the scope the following claims.

Compositions were stored in airtight bottles at 25 °C. CFU/ml was determined after 30, 60 and 90 days and compared to the CFU/ml in the identical compositions without deliquescent salt.

Viability is followed by plate counting.
Container is opened and 1 mL of the anhydrous composition is transferred to tubes and 9 mL Maximum Recovery Diluent (Sigma 07233) is added.
The mixture was homogenized.
CFU/mL determined by plate counting on MRS plates.

All compositions comprising deliquescent salts were stable with a log reduction of less than 0.1 in the full test period. For Compositions without deliquescent (controls) the viability decreased with 1 log after 1 month, with about 3 log after 2 months and with about 4 log after 3 months.

TABLE 1
Stability determined as viable strain at time =
0, and 1, 4, 12 and 24 months
respectively. Shown for storage temperature
25 degrees Celsius. Cell counts are measured as
CFU/ml. (Average of a triplet CFU determination)

| | | T = 3 | T = 6 | T = 12 | T = 24 |
|---|---|---|---|---|---|
| Composition | T = 0 | month | months | months | months |
| No. 1 | 7.1 × 10⁷ | 1.5 × 10⁷ | 2.4 × 10⁷ | 8.6 × 10⁶ | 9.7 × 10⁶ |
| Control | 1.1 × 10⁸ | 6.7 × 10⁵ | 23 | 0 | 0 |
| composition | | | | | |
| No. 1 without | | | | | |
| deliquescent salt | | | | | |
| No. 3 | 5.8 × 10⁹ | 1.5 × 10⁹ | 7.4 × 10⁸ | 9.0 × 10⁷ | 2.2 × 10⁷ |
| Control | 5.5 × 10⁹ | 7.7 × 10⁷ | 2.5 × 10⁴ | 4 | 0 |
| composition | | | | | |
| No. 3 without | | | | | |
| deliquescent salt | | | | | |
| No. 4 | 1.4 × 10⁹ | 1.1 × 10⁹ | 1.0 × 10⁹ | 8.3 × 10⁸ | 3.0 × 10⁸ |
| Control | 1.0 × 10⁹ | 8.4 × 10⁷ | 1.1 × 10⁴ | 1.4 × 10² | 0 |
| composition | | | | | |
| No. 4 without | | | | | |
| deliquescent salt | | | | | |

Control is the identical composition but without the deliquescent salt.

TABLE 2
Stability determined as viable strain at time =
0, and 3, 6, 12 and 24 months
respectively. Shown for storage temperature
25 degrees Celsius. Cell counts are measured
as CFU/ml. (Average of a triplet CFU determination)

| | | T = 3 | T = 6 | T = 12 | T = 24 |
|---|---|---|---|---|---|
| Composition | T = 0 | month | months | months | months |
| No. 2 | 1.9 × 10⁸ | 1.5 × 10⁸ | 1.4 × 10⁸ | 8.6 × 10⁷ | 2.4 × 10⁷ |
| Control | 2.0 × 10⁸ | 7.7 × 10⁷ | 0 | 0 | 0 |
| composition | | | | | |
| No. 2 without | | | | | |
| deliquescent salt | | | | | |
| No. 5 | 1.1 × 10⁹ | 9.2 × 10⁸ | 4.4 × 10⁸ | 2.2 × 10⁸ | 5.7 × 10⁷ |
| Control | 1.3 × 10⁹ | 1.6 × 10⁵ | 0 | 0 | 0 |
| composition | | | | | |
| No. 5 without | | | | | |
| deliquescent salt | | | | | |
| No. 6 | 6.1 × 10⁸ | 4.6 × 10⁸ | 3.8 × 10⁸ | 2.0 × 10⁸ | 6.9 × 10⁷ |
| Control | 5.7 × 10⁸ | 2.3 × 10⁴ | 0 | 0 | 0 |
| composition | | | | | |
| No. 6 without | | | | | |
| deliquescent salt | | | | | |
| No. 7 | 4.4 × 10⁸ | 1.1 × 10⁸ | 9.2 × 10⁷ | 1.0 × 10⁸ | 8.9 × 10⁷ |
| Control | 3.9 × 10⁸ | 8.2 × 10⁶ | 1.1 × 10⁴ | 5 | 0 |
| composition | | | | | |
| No. 7 without | | | | | |
| deliquescent salt | | | | | |

### REFERENCES

Favaro-Trindade et al., (2011) CAB Reviews: Perspectives in Agriculture, Veterinary Science, Nutrition and Natural Resources 6:1-8
WO18185432

1. A topical anhydrous composition comprising an anhydrous substance, at least one viable microorganism and a deliquescent substance.

2. The composition according to claim 1, wherein the weight ratio of the deliquescent substance to the viable microorganism is in the range from 10:1 to 1:10.

3. The topical anhydrous composition according to claim 1, wherein the viable microorganism is present in a concentration above 10⁴ CFU per gram.

4. The composition according to ag claim 1, wherein the deliquescent substance is a salt that adsorbs water at 25 °C, preferably the absorption of water increases the weight of the deliquescent substance by at least 5% by weight (w/w) of the total weight of the absorbed deliquescent substance, more preferably by at least 15% (w/w), more preferably by at least 30% (w/w), more preferably by 50% (w/w).

5. The composition according to claim 1, wherein the deliquescent substance is a salt selected from a water soluble inorganic salt or a water soluble organic salt, preferably, the salt is a water soluble inorganic salt.

6. The composition according to claim 1, wherein the deliquescent substance is a salt selected from the group consisting of calcium chloride (CaCl₂), magnesium chloride (MgCl₂), zinc chloride (ZnCl₂), ferric chloride (FeCl₃), lithium chloride (LiCl), carnallite (KCl·MgCl₂), potassium carbonate (K₂CO₃), sodium acetate anhydrous (CH₃COONa), ammonium nitrate (NH₄NO₃), potassium hydroxide (KOH), dipotassium phosphate (K₂HPO₄), magnesium nitrate (Mg(NO₃)₂), sodium formate (NaCHO₂), sodium acetate (NaC₂H₃O₂), trisodium phosphate (Na₃PO₄), magnesium sulfate (MgSO₄), sodium sulfate (Na₂SO₄), aluminum potassium sulfate (CH₃NH₂Al(SO₄)₂), Manganese(II) chloride (MnCl₂), aluminum sulphate (Al₂(SO₄)₃), aluminum chloride (AlCl₃), cobalt(II) chloride (CoCl₂), sodium alum, NaAl(SO₄)₂, sodium hydroxide (NaOH), and a combination hereof.

7. The composition according to claim 1, wherein the deliquescent substance is a deliquescent salt, preferably the deliquescent salt is selected from magnesium chloride (MgCl₂), calcium chloride (CaCl₂), carnallite (KCl·MgCl₂), potassium carbonate (K₂CO₃), sodium acetate anhydrous (CH₃COONa), and dipotassium phosphate (K₂HPO₄) or any combinations hereof.

8. The composition according to claim 1, wherein the anhydrous substance comprises a vegetable oil and/or a vegetable fat and/or a hydrogenated oil and/or a vegetable wax and/or pasty fatty compound (preferably a vegetable pasty fatty compound).

9. The composition according to claim 1, wherein the viable microorganism is a lyophilized microorganism.

10. The composition according to claim 1, wherein the viable microorganism is a bacterium or a mixture of bacterial strains.

11. The composition according to claim 1, wherein the microorganism is lyophilized and distributed in the composition in lyophilized crystals of a size less than 100 μm.

12. The composition according to claim 1, which comprises, by weight of the total weight of the composition:
50% to 99.5% by weight, preferably 60% to 99% by weight, of at least one anhydrous substance, preferably chosen from vegetable oils, vegetable waxes, vegetable pasty fatty compounds and their mixtures;
0.001% to 20% by weight, preferably 0.005% to 10% by weight, preferably 0.01% to 5% by weight, of at least one viable microorganism;
0.2% to 2% by weight, preferably 0.2% to 1% by weight of at least one deliquescent substance; and
optionally 0.01% to 2% by weight, preferably 0.02% to 1% by weight of at least one antioxidant.

13. The composition according to claim 1, wherein the anhydrous composition is water free or essential water free and wherein water content is below 3% (w/w) relative to the anhydrous composition; such as below 2% (w/w); e.g. below 1% (w/w); such as below 0.5% (w/w); e.g. below 0.1% (w/w); such as below 0.05% (w/w); e.g. below 0.01% (w/w).

14. Method for using a composition according to claim 1 on skin or mucous membranes or genitals of a mammal.

15. A composition comprising the topical anhydrous composition according to claim 1 for use in a cosmetic, in a prophylaxis medicament, for the treatment of a disease, dysfunction or disorder.

16. An anhydrous topical composition comprising a deliquescent substance to stabilize viable microorganisms.

17. A method for providing a topical anhydrous composition comprising a anhydrous substance, at least one viable microorganism and a deliquescent substance wherein the deliquescent substance is subjected to a step of dehydration or partly dehydration before being mixed into the anhydrous substance providing the anhydrous composition.

18. A process for producing an anhydrous composition, the method comprises the steps of:
Providing an anhydrous substance;
Add a deliquescent substance, like a deliquescent salt to the anhydrous substance;
Homogenize the mixture of anhydrous substance and deliquescent substance;
Optionally, cool the melted anhydrous substance comprising the deliquescent substance, like a deliquescent salt, to a temperature below 30 °C:
Add a at least one probiotic microorganism; and
homogenize the mixture providing the anhydrous composition.
